(19) United States
(12) Patent Application Publication
(10) Pub. No.: US 2020/0360060 A1
(43) Pub. Date: Nov. 19, 2020

(54) SPINE FIXING DEVICE HAVING ANTI-SCREW SEPARATION FUNCTION

(71) Applicant: Medyssey Co., Ltd., Chungcheongbuk-do (KR)

(72) Inventors: Jang Jong Wuk, Seoul (KR); Lim Kwun Mook, Gyeonggi-do (KR); Jang Yong, Gyeonggi-do (KR)

(73) Assignee: Medyssey Co., Ltd., Chungcheongbuk-do (KR)

(21) Appl. No.: 16/574,788

(22) Filed: Sep. 18, 2019

(30) Foreign Application Priority Data
May 16, 2019 (KR) 10-2019-0057353

(51) Int. Cl. A61B 17/70; A61B 17/80

(52) U.S. Cl. CPC A61B 17/7059; A61B 17/8042

(57) ABSTRACT

The present invention relates to a spine fixing device having an anti-screw separation function, which includes a fixing plate having a plurality of coupling holes, and screws fastened in the coupling holes and fixing the fixing plate to the spine, in which fixing holes are formed at sides of the coupling holes of the fixing plate, and wing covers inserted and fixed in the fixing holes and having wings to cover heads of the screws and prevent separation of the screws are further included.

### CROSS REFERENCE TO RELATED APPLICATION

The present application claims priority to Korean Patent Application No. 10-2019-0057353, filed May 16, 2019, the entire contents of which is incorporated herein for all purposes by this reference.

### BACKGROUND OF THE INVENTION

### Field of the Invention

The present invention relates to a spine fixing device and, more particularly, to a spine fixing device having an anti-screw separation function which prevents separation of screws and has improved durability and functionality improved by including wing covers for preventing movement or separation from a fixing plate.

### Description of the Related Art

In general, the spine is composed of twenty four bones (except for the sacrum), the bones are connected through joints, and a disc for absorbing a shock and supporting a load every time the human body is moved or the joints are operated is positioned between each the joint.

By this configuration, the spine plays an important role including not only absorbing shock and supporting the posture of a human, but also being a base of motions, and protecting all of the internal organs.

However, when the spine is injured or twisted due to external artificial factors, degeneration, or continuation of abnormal postures for a long period of time, nerves passing through the spinal canal are pressed, so a severe pain is caused.

That is, a patient with a portion of the spine injured cannot function normally, and even if the injury is not severe, the injured portion of the spine is pressed by other adjacent portion or comes in contact with the portion, so pain is caused.

In such circumstances it is required to attach a spine fixing device, which is composed of a spine fixing plate and screws for fixing the spine fixing plate to the spine, to a broken or injured portion of the spine so that the injured portion is not pressed.

However, existing spine fixing devices have a problem in that back-out in which screws are loosened and pulled out backward due to movement or action after a spine fixing plate is fixed to the spine frequently occurs.

Studies about such spine fixing devices have addressed minimizing such back-out, and as a relevant prior art, there is Korean Patent No. 10-0974498, titled “CERVICAL PLATE”.

The prior art relates to a cervical fixing device including a plate having a curvature to be fitted on a plurality of cervical vertebrae, a plurality of holes formed in the plate to correspond to predetermined portions of the cervical vertebrae, and a plurality of screws inserted in the plurality of holes to be fixed to the predetermined portions of the cervical vertebrae. The cervical fixing device includes one or more cap insertion holes formed between the plurality of holes on the top of the plate and one or more elastic caps respectively inserted in the one or more cap insertion holes to prevent separation of the screws from the plurality of holes, and is characterized in that the one or more elastic caps each have an anti-separation protrusion extending a predetermined length over one of the plurality of holes and elastically move in the anti-separation protrusion.

According to the prior art, screws can be inserted with the caps fastened, so the caps can be fastened in advance to the plate before a surgical operation or the device is manufactured, so there is no additional work for fastening the caps during a surgical operation. Accordingly, surgical operation time can be reduced and surgical operation accuracy can be improved.

However, according to the prior art, since the elastic caps are provided to prevent separation of the screws from the holes and the anti-separation protrusions are formed by extending the end portions of the elastic caps, the anti-separation protrusions hold the heads of the screws, thereby preventing separation of the screws from the holes.

However, according to this structure, since only the heads of the screws are partially held, it is impossible to completely prevent separation of the screws from the holes. Further, since the screws are pressed in only one direction, there is a problem in that the direction of the screws may be changed or the possibility of separation is high after the direction of the screw is changed, so the spine is not fixed well.

Further, according to the prior art, the cap insertion holes for inserting the elastic caps are formed and the elastic caps are repeatedly formed in a U-shape or a V-shape and have an anti-separation protrusion at the end, so there is a problem in that the entire structure is complicated and manufacturing is difficult.

Further, there are other fixing methods that provide a separate plate on a plate to prevent separation of screws or use locking threads on screws and the heads of a plate.

However, these fixing methods also have a problem in that the plate cover is separated, or in the method that uses locking threads, since the screws and the plate are fixed, separation of the screws can be prevented, but the load of the entire spine concentrates on the plate and the screws, so the fatigue failure of the device is accelerated.

### SUMMARY OF THE INVENTION

The present invention has been made in an effort to solve the problems and an object of the present invention is to provide a spine fixing device having an anti-screw separation function which prevents separation of screws and has improved durability and functionality improved by including wing covers for preventing movement or separation from a fixing plate.

In order to achieve the objective, a spine fixing device having an anti-screw separation function includes a fixing plate having a plurality of coupling holes, and screws fastened in the coupling holes and fixing the fixing plate to the spine, in which fixing holes are formed at sides of the coupling holes of the fixing plate, and wing covers inserted and fixed in the fixing holes and having wings to cover heads of the screws and prevent separation of the screws are further included.

The wing cover may have: a body being is inserted and fixed in the fixing hole of the fixing plate; and wings formed on the body and horizontally extending to cover the head of the screw.

A locking step may be formed at a lower end of the body so that the body is not separated upward after inserted in the fixing hole of the fixing plate.

One or more cut portions may be vertically formed on the body so that an outer diameter of the body is elastically changed.

A supporting portion having an outer diameter larger than an inner diameter of the fixing hole of the fixing plate may be formed at an upper end of the body.

A bump may be formed at a side of the supporting portion to be locked to the fixing plate, thereby preventing unexpected rotation of the wing cover.

Fastening portions may be formed inside the body so that the wing cover is easily fastened to the fixing plate.

A tapered portion may be formed at an end portion of the wing.

The wings may be formed in pair with a predetermined angle from the body to cover simultaneously a pair of screws.

Guide portions accommodating and fixing the wing covers may be formed on the fixing plate around the fixing holes to correspond to a rotational radius of the wing covers.

Bump grooves in which the bumps for preventing unexpected rotation of the wing covers may be formed at sides of the guide portions.

The present invention has an effect of preventing separation of screws and improving durability and functionality of a spine fixing device by including the wing covers to prevent separation of screws from the fixing plate

Further, according to the spine fixing device of the present invention, since movement of the screws fastened to the fixing plate is prevented by the wing covers, the fixing plate is stably coupled treatment or correction of spine diseases is achieved well.

Further, since the wing cover according to the present invention can prevent movement of the screw only by being inserted and rotated in the fixing hole of the fixing plate, there is an effect in that surgical operation time is reduced and surgical operation accuracy is improved.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a perspective view of a spine fixing device having an anti-screw separation function according to the present invention;

FIG. 2 is an exploded perspective view of the spine fixing device having an anti-screw separation function according to the present invention;

FIGS. 3A and 3B are respectively a top perspective view and a bottom perspective view of a wing cover according to the present invention;

FIG. 4 is a vertical view of the spine fixing device having an anti-screw separation function according to the present invention (A-A′ in FIG. 1); and

FIGS. 5A and 5B are schematic views showing the operation of the spine fixing device having an anti-screw separation function according to the present invention;

### DETAILED DESCRIPTION OF THE INVENTION

The present invention relates to a spine fixing device and, more particularly, to a spine fixing device having an anti-screw separation function which prevents separation of screws and has improved durability and functionality improved by including a wing cover for preventing movement or separation from a fixing plate.

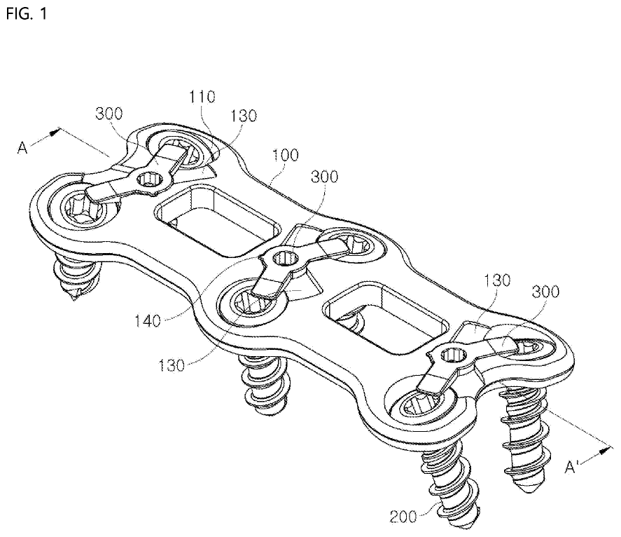

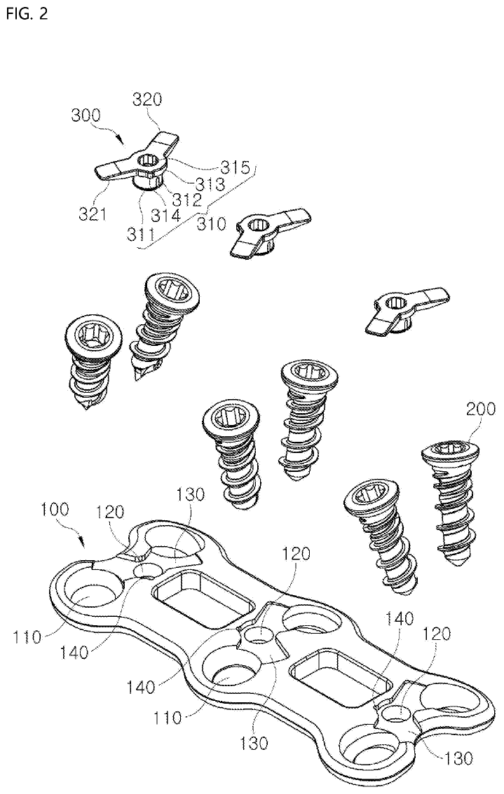

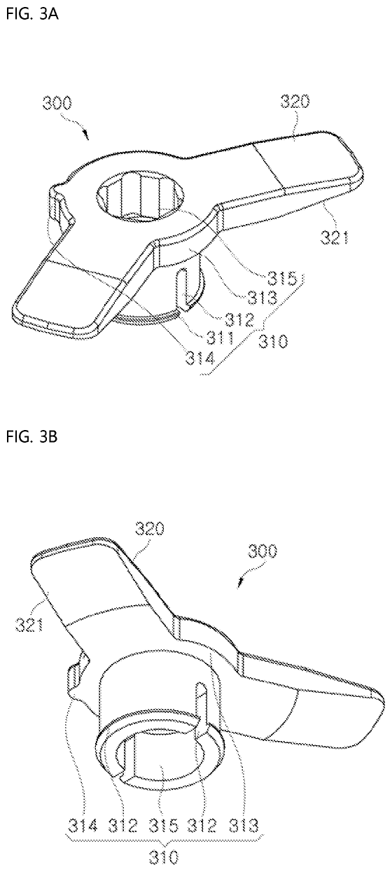

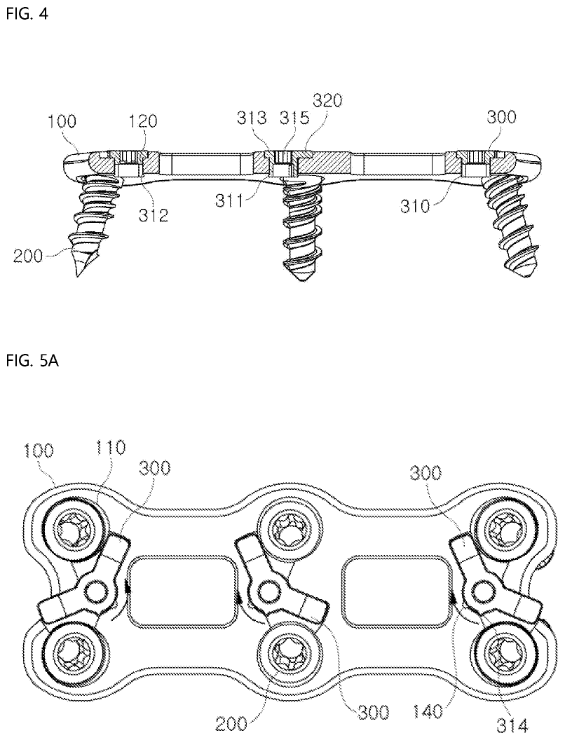

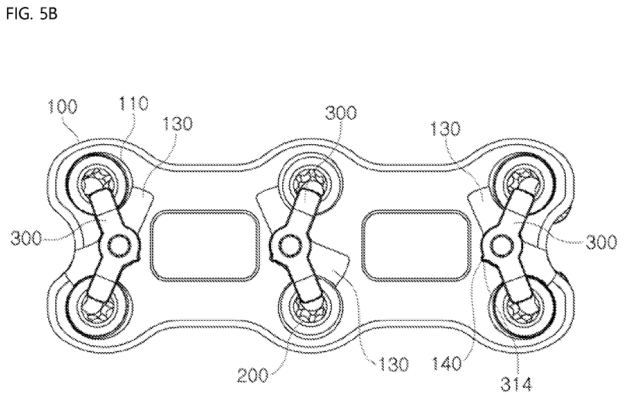

Embodiments of the present invention will be described hereafter in detail with reference to the accompanying drawings. FIG. 1 is a perspective view of a spine fixing device having an anti-screw separation function according to the present invention, FIG. 2 is an exploded perspective view of the spine fixing device having an anti-screw separation function according to the present invention, FIGS. 3A and 3B are respectively a top perspective view and a bottom perspective view of a wing cover according to the present invention, FIG. 4 is a vertical view of the spine fixing device having an anti-screw separation function according to the present invention (A-A′ in FIG. 1), and FIGS. 5A and 5B are schematic views showing the operation of the spine fixing device having an anti-screw separation function according to the present invention.

As shown in the figures, a spine fixing device according to the present invention includes a fixing plate 100 having a plurality of coupling holes 110; and screws 200 fastened in the coupling holes 110 and fixing the fixing plate 100 to the spine, and is characterized in that fixing holes 120 are formed at sides of the coupling hole 110 of the fixing plate 100, and wing covers 300 that are inserted and fixed in the fixing holes 120 and have wings 320 to cover the heads of the screws 200 and prevent separation of the screws 200 are further included.

The spine fixing device according to the present invention is a device for performing treatment or correction on various spinal diseases (an intervertebral disc disease, a dislocation, a fracture, deformation, etc.) by minimizing movement of vertebrae by connecting and fixing adjacent vertebrae.

First, in the spine fixing device according to the present invention, the fixing plate 100 is formed in a size that can cover a plurality of vertebrae, that is, at least two or more vertebrae, and is generally formed in a shape with a recessed inner side (part that is brought in contact with vertebrae) to correspond to the shape of the spine to be operated.

Further, the fixing plate 100 is made of a human-friendly material such as titanium, a titanium alloy, or stainless steel.

Further, the fixing plate 100 may have surgical operation holes to show the surgical operation situation of connection portions of each vertebra and bones can be inserted, that is, surgical operation holes may be formed in various shapes and sizes in the fixing plate 100 according to the present invention except for the areas where the coupling holes 110, the fixing holes 120, and guides 130 are formed.

A plurality of coupling holes 110 is formed in the fixing plate 100, so the screws 200 are inserted therein and fix the fixing plate 100 to the spine. They are formed to be able to connect a fix at least two or more vertebrae.

The coupling holes 110 are formed through the fixing plate 100, so when the screws 200 are inserted therein and driven into vertebrae, the heads of the screws 200 are seated in the coupling holes 110. As shown in the figures, in an embodiment of the present invention, a total of six coupling holes 110 are provided and screws 200 are fastened in the coupling holes 110, respectively.

The coupling holes 110 are formed to correspond to the shape of the heads of the screws 200 such that the diameter decreases downward, so the screws 200 can be fixed in the fixing plate 100 without being fully driven in bones and the angle of the screws 200 can be easily adjusted.

Further, the fixing holes 120 are formed at sides of the coupling holes 110 in the fixing plate 100. The fixing holes 120 are formed to fix the wing covers 300 of the present invention to be described below and are formed up and down through the fixing plate 200, and one fixing hole 120 is provided for each pair of the coupling holes 110, adjacent to the coupling holes 110 in which the screws 200 are fastened.

This is for making it possible to fix two screws 200 at a time with a wing cover 300 of the present invention. That is, according to an embodiment of the present invention, since a total of six coupling holes 110 and a total of six screws 200 are provided, three fixing holes 120 are formed each at the same distance from two coupling holes 110.

The wing covers 300 are inserted and fixed in the fixing holes 120 of the fixing plate 100 and have wings 320 to cover the heads of the screws 200, thereby preventing separation of the screws 200. That is, the wing covers 300 extend in a wing shape to be able to cover the heads of the screws 200, and can be inserted in the fixing holes 120. Further, the wing covers 300 may be formed to have two wings making a predetermined angle from the fixing holes 120.

In the present invention, the wing covers 300 prevent the screws 200 from moving or unexpectedly separating so that the fixing plate 100 is stably fixed to vertebrae even by movement of a body or an external shock. To this end, it may be possible to simply cover the heads of the screws 200, press the screws 200, or change the pressing force in accordance with the angle of the heads of the screws 200.

Since the screws 200 are individually fixed by the wing covers 300 having the simple structure, stress in the device is distributed, thereby minimizing fatigue of the device. Further, the screws 200 are stably fixed, so movement or unexpected separation of the screws 200 is prevented.

The wing covers 300 according to the present invention each have a body 310 that is inserted and fixed in the fixing hole 120 of the fixing plate 100, and wings 320 formed on the body 310 and horizontally extending to cover the head of the screw 200.

That is, the wing cover 300 has a body 310 having a shape corresponding to the fixing hole 120, that is, a cylindrical shape to be able to be inserted and fixed in the fixing hole 120, and wings 320 formed on the body 310 and extending a predetermined length from the body 310 to be able to cover the head of the screw 200.

The wings 320 may be formed in pair with a predetermined angle from the body 310 to cover simultaneously a pair of screws 200 around the fixing hole 120, as described above.

That is, the length and angle of the wings 320 are determined in accordance with the gap between screws 200 to be fixed simultaneously. Further, the wings 320 are formed in a length to be able to cover only portions of the heads of the screws 200 so that even if the heads of the screws 200 somewhat protrude, the screws 200 can be covered by the wing cover 300 (wing). Further, the wings 320 have a predetermined angle from the body 310 so that force is distributed stably and in balance throughout the wing cover 300.

Further, when the screws 200 are abnormally fastened or the heads protrude, the wing covers 300 may not be rotated. Accordingly, a tapered portion 321 is formed at the end portion of the bottom of the wing 320 so that the wing 320 can be rotated over the head of the screw 200. That is, the wing 320 can be somewhat bent when it is rotated by the tapered portion 321.

The tapered portion 321 may be formed at the lower end portion of the bottom of the wing 320 that is brought in contact with the head of the screw 200, and if necessary, the tapered portion 321 may be formed also at the end portion of the top so that the wing 320 can be further bent.

Further, a locking step may be formed at the lower end of the body 310 so that the body 310 is not separated upward after inserted in the fixing hole 120 of the fixing plate 100.

That is, the body 310 of the wing cover 300 is formed in a rivet shape so that the locking step 311 is locked to the opposite side (bottom) of the fixing plate 100 after the body 310 is inserted in the fixing hole 120. Accordingly, the wing cover 300 inserted in the fixing hole 120 is not unexpectedly separated upward from the fixing plate 100.

In order to easily fit the body 310 of the wing cover 300 into the fixing hole 120, one or more cut portions 312 are vertically formed on the body 310, so when the body 310 is fitted in the fixing hole 120, the body is forcibly fitted by the locking step 311 and the outer diameter of the body 310 changes (decreases), whereby the body 310 is easily inserted and fitted. Further, when the body 310 is completely fitted in the fixing hole 120, the locking step 311 reaches the opposite side of the fixing plate 100 and the outer diameter of the body 310 returns to the initial state. Accordingly, the wing cover 300 is stably fixed without unexpectedly separating upward by the locking step 311.

Further, a supporting portion 313 having an outer diameter larger than the inner diameter of the fixing hole 120 of the fixing plate 100 is formed at the upper end of the body 310, so the wing cover 300 is prevented from unexpectedly moving up and down respectively by the supporting portion 313 and by the locking step 311 with respect to the fixing hole 120, so it is stably supported and fixed.

Further, a bump 314 is formed at a side of the supporting portion 313 to be locked to the fixing plate 100, thereby preventing unexpected rotation of the wing cover 300.

That is, the bump 314 protrudes from the supporting portion 313 and is locked to the fixing plate 100 to prevent more rotation, and the bump 314 may be locked in a bump groove 140 of the fixing plate 100.

Further, fastening portions 315 are formed inside the body 310 so that the wing cover 300 is easily fastened (fitted and rotated) to the fixing plate 100.

As described above, the wing cover 300 is inserted and fixed in the fixing hole 120 formed in the fixing plate 100 to be able to cover the head of the screw 200, thereby preventing unexpected separation, rotation, and movement of the screw 200.

The wing cover 300 according to the present invention is fitted in the fixing hole 120 of the fixing plate 100 and guide portions 130 accommodating and fixing the wing covers 300 may be further formed on the fixing plate 100 around the fixing holes 120 to correspond to the rotational radius of the wing covers 300.

The guide portion 130 is formed in a groove shape such that the wing cover 300 is accommodated and seated downward and can be rotated with a predetermined rotational radius on the top of the fixing plate 100 and can stop rotating after being rotated at a predetermined angle.

That is, the guide portion 130 is formed in a shape that allows the wing cover 300 to be seated (fitted in the fixing hole 120) and to be fixed after being rotated. When the wing cover 300 is inserted into the fixing hole 120 and seated in the guide portion 130, and is then rotated to cover the head of the screw 200, the wing cover 300 is rotated within a predetermined rotational radius not to further rotate.

In this case, as described above, the bump 314 for preventing unexpected rotation of the wing cover 300 is fitted in the bump groove 140 formed on the fixing plate 100, so movement and rotation of the wing cover 300 are prevented by the guide portion 130, the bump 314, the supporting portion 313, and the locking step 311, thereby preventing movement of the screw 200 by stably cover the head of the screw 200, that is, pressing the screw 200.

FIGS. 5A and 5B are schematic views showing the operation of the spine fixing device having an anti-screw separation function according to the present invention.

When the screw 200 is relatively normally fastened, the wing cover 300 can directly cover (press) the head of the screw 200 when the wing cover 300 is inserted and fixed in the fixing hole 120. However, as shown in FIG. 5A, it may be possible to insert and fix the wing cover 300 into the fixing hole 120 such that the wing cover 300 is seated in the guide portion 130, and then, rotate the wing cover 300, as shown in FIG. 5B, to press the head of the screw 200.

Further, even if the screw 200 is abnormally fastened, as shown in FIGS. 5A and 5B, the wing cover 300 is inserted and then fixed in the fixing hole 120 such that the wing cover 300 is seated in the guide portion 130 (FIG. 5A), and then the wing cover 300 is rotated, thereby pressing the head of the screw 200 (FIG. 5B).

When the head of the screw 200 is abnormally positioned or the head of the screw 200 protrudes, the wing cover 300 is rotated over the head of the screw 200 by the tapered portion 321 of the wing cover 300, whereby the wing cover 300 presses and fixes the screw 200 with appropriate force.

In this case, the wing cover 300 is rotated and stopped at an appropriate position and presses the head of the screw 200. Further, the wing cover 300 is fixed without further rotating by the guide portion 130 and the bump 314.

As described above, the spine fixing device according to the present invention is composed of a fixing plate, screws, and wing covers in a broad meaning, and movement of the screws fastened to the fixing plate is prevented by the wing covers, thereby preventing separation of the screws. Accordingly, durability and functionality of the spine fixing device are improved and treatment or correction of spine diseases is achieved well by stably coupling the fixing plate.

Further, the wing cover according to the present invention can prevent movement of the screw only by being inserted and rotated in the fixing hole of the fixing plate, thereby reducing surgical operation time and improving surgical operation accuracy.

What is claimed is:

1. A spine fixing device having an anti-screw separation function, which comprises a fixing plate having a plurality of coupling holes, and screws fastened in the coupling holes and fixing the fixing plate to the spine,
wherein fixing holes are formed at sides of the coupling holes of the fixing plate, and wing covers inserted and fixed in the fixing holes and having wings to cover heads of the screws and prevent separation of the screws are further included.

2. The spine fixing device of claim 1, wherein the wing cover has:
a body being is inserted and fixed in the fixing hole of the fixing plate; and
wings formed on the body and horizontally extending to cover the head of the screw.

3. The spine fixing device of claim 2, wherein a locking step is formed at a lower end of the body so that the body is not separated upward after being inserted in the fixing hole of the fixing plate.

4. The spine fixing device of claim 3, wherein one or more cut portions are vertically formed on the body so that an outer diameter of the body is elastically changed.

5. The spine fixing device of claim 2, wherein a supporting portion having an outer diameter larger than an inner diameter of the fixing hole of the fixing plate is formed at an upper end of the body.

6. The spine fixing device of claim 5, wherein a bump is formed at a side of the supporting portion to be locked to the fixing plate, thereby preventing unexpected rotation of the wing cover.

7. The spine fixing device of claim 2, wherein fastening portions are formed inside the body so that the wing cover is easily fastened to the fixing plate.

8. The spine fixing device of claim 2, wherein a tapered portion is formed at an end portion of the wing.

9. The spine fixing device of claim 2, wherein the wings are formed in pair with a predetermined angle from the body to cover simultaneously a pair of screws.

10. The spine fixing device of claim 1, wherein guide portions accommodating and fixing the wing covers are formed on the fixing plate around the fixing holes to correspond to a rotational radius of the wing covers.

11. The spine fixing device of claim 10, wherein bump grooves in which the bumps for preventing unexpected rotation of the wing covers are formed at sides of the guide portions.
